(19) United States
(12) Patent Application Publication
(10) Pub. No.: US 2014/0328892 A1
(43) Pub. Date: Nov. 6, 2014

(54) USE OF ALFIMEPRASE IN THE TREATMENT OF THROMBOSIS

(71) Applicant: Jullia Y. Lee, Ann Arbor, MI (US)

(72) Inventor: Jullia Y. Lee, Ann Arbor, MI (US)

(21) Appl. No.: 14/245,110

(22) Filed: Apr. 4, 2014

Related U.S. Application Data
(60) Provisional application No. 61/808,423, filed on Apr. 4, 2013.

(51) Int. Cl. A61K 38/48; A61M 37/00; A61K 31/4365; A61N 5/06; C12N 9/50; A61K 45/06

(52) U.S. Cl. CPC A61K 38/4886; C12N 9/50; A61K 45/06; A61K 31/4365; A61N 5/062; A61M 37/0092; A61N 2005/067
USPC 424/422; 424/94.67; 424/133.1; 604/20; 604/22

(57) ABSTRACT

Methods for treatment of arterial or venous thrombosis in patients undergoing an endovascular intervention or a surgical procedure, and more particularly, administering alfimeprase directly into the thrombus or adjacent to the thrombus in a coronary, cerebral, or peripheral vessel.

### CROSS-REFERENCE TO RELATED APPLICATION

This application claims the benefit of U.S. Patent Application No. 61/808,423, filed Apr. 4, 2013, which is incorporated herein by reference in its entirety.

### FIELD

The present invention relates generally to treatment of arterial or venous thrombosis in patients using catheter based or surgical procedures and more particularly to use of alfimeprase administered directly into the thrombus or adjacent to the thrombus in a coronary or cerebral vessel.

### BACKGROUND

Atherosclerosis and cardiovascular disease are leading causes of mortality and morbidity worldwide. Each process can affect major and minor arterial vessels. Yet while arterial and venous thrombotic disorders have numerous origins it is at the end organs where the effects of the thrombosis are most evident and where those effects result in clinical manifestation. Arterial thrombosis, for example, may manifest as sudden cardiac death, acute coronary syndromes (ACS), stroke, or peripheral embolization. Venous thrombosis may manifest as acute deep vein thrombosis (DVT), pulmonary embolism (PE), or paradoxical arterial embolization.

The underlying causes of these manifestations range from atherosclerosis due to plaque rupture or erosion (e.g., sudden death, ACS, etc.), cardiac embolization from atrial fibrillation or left ventricular aneurysm (often secondary to coronary atherosclerosis), stasis, and immobility (e.g., postoperative DVT), hypercoagulable state (activated protein C deficiency, malignancy), and a variety of rare disorders. Furthermore, thrombosis may complicate the performance of cardiovascular procedures or initiate malfunction of foreign devices implanted in the cardiovascular system valves, arterial stents, venous filters, bypass grafts, etc.

ST elevation myocardial infarction (STEMI) is the most dramatic presentation of atherothrombosis and arises due to a thrombotic occlusion of a coronary artery. The underlying etiology of STEMI is usually plaque rupture or erosion with subsequent formation of a red clot and acute total or near obstruction of coronary flow to the downstream myocardial bed. Accordingly, the goal of therapy for treatment of STEMI has been restoration of myocardial perfusion using either systemic thrombolytic agents or primary percutaneous coronary intervention (PCI). Since primary PCI is more effective at restoring blood flow, and is associated with a reduction in mortality and other major complications such as stroke, it has emerged as the pre-eminent therapy for patients with STEMI. Current guidelines from the American College of Cardiology/American Heart Association and the European Society of Cardiology support primary PCI as the preferred therapy in patients with STEMI if it can be provided in an expedited fashion.

Primary PCI usually involves either removal of the thrombus with a thrombectomy device, or disruption of the occlusion using balloon angioplasty or stents. Primary PCI is highly successful in ensuring restoration of coronary flow but this does not always result in normalization of myocardial perfusion. Lack of myocardial reperfusion results in diminished (or absent) myocardial salvage, adverse remodeling, reduced left ventricular function, and increased likelihood of congestive heart failure and death on long term follow up. The angiographic hallmark of this phenomenon is lack of myocardial blush, although the lack of complete ST resolution appears to be a more sensitive and specific marker of myocardial reperfusion.

The institution of systems for rapid provision of primary PCI has resulted in a dramatic reduction in short-term mortality associated with STEMI. The failure of complete myocardial salvage however, continues to impact the long-term health outcome of these patients and contributes to the growing epidemic of congestive heart failure. In a recent study of 1,406 patients undergoing primary PCI in Germany, no-reflow phenomenon was diagnosed in 410 patients (29%). Infarct size was 15.0% (6.0% to 29.0%) of the left ventricle in the no-reflow group versus 8.0% (2.0% to 21.0%) of the left ventricle in the reflow group (p<0.001). Over a 5 year follow up period, there were 132 deaths with 59 deaths among patients with no-reflow and 73 deaths among patients with reflow (Kaplan-Meier estimates of 5-year mortality 18.2% and 9.5%, respectively; odds ratio: 2.02; 95% confidence interval: 1.44 to 2.82; p<0.001). Other studies have demonstrated similarly worse course of patients who have no-reflow after undergoing primary PCI.

The recognition of importance of achieving myocardial reperfusion has generated multiple studies to explore various strategies to prevent no-reflow phenomenon. The use of intracoronary adenosine or strategies such as post-conditioning or remote conditioning has not demonstrated any benefit when tested in clinical trials. The only therapy that has been demonstrated to reduce the incidence of no-reflow has been use of aspiration thrombectomy. In various studies and meta-analysis, there has been a consistent improvement in myocardial salvage, ST resolution, improved angiographic success rate, and a trend towards reduction in mortality in patients who are randomized to aspiration thrombectomy. However, aspiration thrombectomy is not universally successful in restoration of normal myocardial perfusion and prevention of coronary no-reflow phenomenon. In the meta-analysis by Tamhane and colleagues, ST resolution occurred in 923/1500 patients undergoing thrombectomy versus 715/1494 in the control arm. While better than control PCI, thrombectomy is still associated with a significant proportion of patients not achieving optimal myocardial reperfusion. In an elegant follow up study from the TAPAS trial, Fokkema and colleague evaluated the presence of angiographically visible distal embolization in patients undergoing primary PCI. Distal embolization was detected in 6% of the patients and was associated with poorer ST resolution, larger infarct size, and a higher incidence of death and myocardial infarction.

There is thus an unmet clinical need to further enhance outcome in patients with STEMI.

A similar challenge is associated with treatment of ischemic stroke. Ischemic stroke is either related to embolization or thrombosis and is generally treated with thrombolytic therapy. The results with endovascular therapy of stroke remain relatively poor and multiple therapies are being explored to improve outcomes of these devices. Again, a major challenge in endovascular therapy of stroke remains distal embolization of thrombus, which results in occlusion of multiple small branches and plugging up of microvessels with clot debris with the resulting loss of cerebral tissue and disability or death.

Similar issues can become relevant when thrombotic lesions are treated elsewhere in the body. Thrombotic lesions can embolize and cause tissue loss in patients with peripheral arterial stenosis such as in vessels supplying the limbs during treatment with an endovascular device such as a stent or balloon or using devices such as laser atherectomy, etc. or when aortic disease such as aortic aneurysms are treated with an endovascular approach. Similar problems are well recognized, by those familiar with the art, in other vascular beds such as renal arteries, mesenteric vessels, or any branch of the aorta or pulmonary artery or the branches thereof.

There has been an increased interest in treatment of deep vein thrombosis with fibrinolysis or ultrasound enhanced thrombolysis. The issues of distal embolization and pulmonary embolization remain equally important in venous beds.

We propose a novel pharmacological regimen that would facilitate local thrombolysis and reduce the risk of no-reflow and distal embolization after percutaneous intervention in patients with myocardial infarction or after other endovascular interventions.

Prior work has established that the culprit thrombus is usually a red clot in patients with no-reflow or in those with angiographically visible distal embolization. This suggests that the use of additional pharmacotherapy that can achieve thrombolysis of the residual clot without increasing risk of bleeding or activating inflammatory cascade would be a viable strategy to reduce no-reflow and enhance myocardial salvage. Prior work using tissue plasminogen activator or streptokinase has paradoxically failed to demonstrate an improvement in outcome in these patients in part due to the increased risk of bleeding and also in part due to activation of complement pathway and increased tissue injury. These agents have been hypothesized to increase the risk of bleeding and inflammation both at the site of the endovascular intervention as well as into the distal bed. There is thus an unmet need to develop a therapeutic strategy to enhance the outcome of percutaneous treatment of arterial thrombosis without an increase in risk of bleeding. In particular, there is a need for a treatment that enhances the outcome of patients who have a STEMI.

### SUMMARY

The present application describes the use of intra-arterial alfimeprase to enhance myocardial salvage (or salvage of the distal target bed) in patients who have angiographic evidence of thrombus or evidence of embolization before or after use of a mechanical endovascular device.

Alfimeprase is a fibrinolytic enzyme that has direct proteolytic activity against the Aα chain of fibrinogen (primarily cleaves at position Lys413Leu414). The clinically available thrombolytic agents such as urokinase and recombinant tissue plasminogen activator (rt-PA or alteplase) are plasminogen activators (PAs) that promote thrombolysis by depending upon the endogenous fibrinolytic system. PAs catalyze the conversion of plasminogen into plasmin, a serine protease that degrades fibrin to achieve clot lysis. Alfimeprase does not rely on the endogenous fibrinolytic system; therefore, alfimeprase can be distinguished from PAs by its unique mechanism of action and is defined as a direct fibrinolytic agent.

Pharmacological in vivo studies have shown that alfimeprase is significantly more effective than plasminogen activators at breaking up thrombi. Alfimeprase does not interact with plasminogen, and its actions are localized due to rapid inactivation by α2-macroglobulin (α2M), a prevalent protease inhibitor. The covalently bound alfimeprase-α2M complex is believed to be cleared by low density lipoprotein receptor-related protein in hepatocytes.

### DETAILED DESCRIPTION OF THE INVENTION

Any reference to administration of alfimeprase is to be understood to refer to administration of an effective amount of alfimeprase. An effective amount may vary according to factors known in the art, such as the disease state, age, sex, and weight of the patient. Determination of a therapeutically effective amount is well within the capabilities of those skilled in the art.

While a number of embodiments of this disclosure are described, it is apparent that the basic examples may be altered to provide other embodiments that use or encompass methods and processes of this invention. The embodiments and examples are for illustrative purposes and are not to be interpreted as limiting the disclosure, but rather, the appended claims define the scope of this invention.

The terms “treatment”, “treating”, “treat”, and the like, refer to obtaining a desired pharmacologic and/or physiologic effect such as mitigating a disorder or condition in a patient. The term “treatment” includes, preventing a disorder from occurring in the patient, particularly when the patient is predisposed to acquiring the disorder, and/or alleviating or reversing the disorder. The term “prevent” does not require that the disorder or condition be completely avoided.

Throughout the description and claims of this specification the word “comprise” and other forms of the word, such as “comprising” and “comprises”, means including but not limited to, and is not intended to exclude, for example, other additives, components, integers, or steps.

As used in the description and the appended claims, the singular forms “a”, “an”, and “the” include plural referents unless the context clearly dictates otherwise. Thus, for example, reference to “a composition” includes one composition or mixtures of two or more such compositions.

PCI is an abbreviation for percutaneous coronary intervention.

STEMI is an abbreviation for ST elevation myocardial infarction.

As used in the description and the appended claims, the use of “or” in a list includes any combination of the items in the list unless the context clearly dictates otherwise. For example, “co-administered with an anti-coagulant or a platelet aggregation inhibitor” includes co-administration with an anti-coagulant, a platelet aggregation inhibitor, or a combination of the two. Likewise, a reference to administration “before” a procedure does not exclude administration during or after a procedure. For example, administration may include administration before, during, and after; before and after only; or before and during only.

The antecedent “about” indicates that the values are approximate. For example for the range from “about 1 mg to about 50 mg” indicates that the values are approximate values. The range of “about 1 mg to about 50 mg” includes approximate and specific values; for example, the range includes 1.0 mg and 50.0 mg.

When a range is described as between two numbers, the range includes both endpoints of the range as well as all numbers in between. For example, between 1 mg and 10 mg includes 1 mg and 10 mg and all amounts between.

Doses are provided as mg per kilogram of the patient weight (mg/kg).

An embodiment of the present invention is a method of treating a patient with an arterial thrombus, comprising administering alfimeprase locally to the thrombus. A particular embodiment of the present invention is a method of treating a patient with an arterial thrombus, wherein the thrombus is in a coronary artery, comprising administering alfimeprase locally to the thrombus. Another embodiment is method of treating a patient with an arterial thrombus, wherein the thrombus is in a cerebral artery, comprising administering alfimeprase locally to the thrombus. A particular embodiment of the invention is a method of treating a patient with an arterial thrombus, wherein the thrombus is in a peripheral vessel, comprising administering alfimeprase locally to the thrombus. Another embodiment of the present invention is a method of treating a patient with a venous thrombus, comprising administering alfimeprase locally to the thrombus. A particular embodiment of the invention is a method of treating a patient with a venous thrombus, wherein the thrombus is in a cerebral vessel, comprising administering alfimeprase locally to the thrombus. A particular embodiment of the invention is a method of treating a patient with a venous thrombus, wherein the thrombus is in a peripheral vessel, comprising administering alfimeprase locally to the thrombus. In general, a patient is determined to have a thrombus based on angiographic evidence of a thrombus or evidence of embolization before or after use of a mechanical endovascular device.

Generally the alfimeprase is administered during an endovascular intervention, including, but not limited to, PCI or removal or partial removal of the thrombus. The alfimeprase is administered at any point during the treatment, for example before the intervention, during the intervention, after the intervention, or any combination of before, during, and after.

When referring to prior or before, which are used interchangeably herein, with respect to the administration of alfimeprase, it is meant that the alfimeprase is administered in conjunction with any method of providing mechanical intervention or delivery of a force such as laser or ultrasound. For example, the alfimeprase may be administered between about 1 hour and about 1 second before endovascular intervention. Or the alfimeprase may be administered between about 30 minutes and about 1 second before endovascular intervention. In other embodiments, the alfimeprase is administered about 20 minutes, or about 15 minutes, or about 10 minutes, or about 5 minutes, or about 1 minute before endovascular intervention.

Likewise, when referring to after with respect to the administration of alfimeprase, it is meant that the alfimeprase is administered in conjunction with any method of providing mechanical intervention or delivery of a force such as laser or ultrasound. For example, the alfimeprase may be administered between about 1 second and about 1 hour after endovascular intervention. Or the alfimeprase may be administered between about 1 minute and about 30 minutes after endovascular intervention. Or the alfimeprase may be administered between about 1 minute and 20 minutes after endovascular intervention. In other embodiments, the alfimeprase is administered for about 20 minutes, or for about 15 minutes, or for about 10 minutes, or for about 5 minutes, or for about 1 minute after endovascular intervention.

A particular embodiment is a method of treating a patient with a coronary arterial thrombus, comprising administering alfimeprase locally to the thrombus, wherein the patient has had a STEMI. Another embodiment is a method of treating a patient with a coronary arterial thrombus, comprising administering alfimeprase locally to the thrombus, wherein the patient has had a STEMI and the patient is being treated by PCI. In certain embodiments, the alfimeprase is administered before PCI, during PCI, after PCI, or any combination of before, during, and after.

In any of the foregoing embodiments, it is contemplated that the patient has had a STEMI. It is further contemplated that in any of the foregoing embodiments, the patient has had a STEMI less than 24 hours before the alfimeprase is administered. In particular embodiments, the alfimeprase is administered to the thrombus in less than 24 hours after the onset of the STEMI, or in less than 18 hours, or in less than 12 hours, or in less than 6 hours, or in less than 5 hours, or in less than 4 hours, or in less than 3 hours, or in less than 2 hours, or in less than 1 hour.

In any of the foregoing embodiments, the alfimeprase may be administered adjacent to the thrombus or the alfimeprase may be administered directly into the thrombus. The alfimeprase can be administered by any suitable method. Suitable methods are well known in the art. Examples include, but are not limited to, delivery of the alfimeprase via infusion wire, via catheter, via balloon, via a drug coated stent, or via a drug coated balloon.

It is contemplated that in any of the foregoing embodiments, the patient is treated using balloon angioplasty. Thus, another embodiment is a method of treating a patient with an arterial thrombus, wherein the patient is treated using balloon angioplasty. In particular embodiments, the alfimeprase is administered before balloon angioplasty, during balloon angioplasty, after balloon angioplasty, or any combination of before, during, and after. In certain embodiments, the balloon is used to administer the alfimeprase such as by a drug coated balloon. In certain embodiments, the drug is delivered via a balloon that infuses the drug from its surface or via an additional port. Another embodiment is a method of treating a patient with a venous or arterial thrombus, wherein the patient is treated using a stent. In certain embodiments, the stent is used to administer the alfimeprase such as by a drug coated stent.

In yet another embodiment is a method of treating a patient with a venous thrombus, wherein the patient is treated using balloon angioplasty. In particular embodiments, the alfimeprase is administered before balloon angioplasty, during balloon angioplasty, after balloon angioplasty, or any combination of before, during, and after. In certain embodiments, the balloon is used to administer the alfimeprase such as by a drug coated balloon. In certain embodiments the drug is delivered via a balloon that infuses the drug from its surface or via an additional port(s). Another embodiment is a method of treating a patient with a venous or arterial thrombus, wherein the patient is treated using a stent. In certain embodiments, the stent is used to administer the alfimeprase such as by a drug coated stent.

Dosing of alfimeprase is well known in the art. Alfimeprase, as used in the embodiments of the present disclosure, is administered at a dose of between about 0.01 mg/kg and about 20 mg/kg. In another embodiment, the dose is between about 0.1 mg/kg and about 20 mg/kg. In a particular embodiment, the dose is between about 0.5 mg/kg and about 20 mg/kg. In yet another embodiment, the dose is between about 0.1 mg/kg and about 10 mg/kg. In still another embodiment, the dose is between about 0.3 mg/kg and about 10 mg/kg. In yet another embodiment, the dose is between about 0.01 mg/kg and about 5 mg/kg. In still another embodiment, the dose is between about 0.025 mg/kg and about 1 mg/kg. In another embodiment, the dose is between about 0.05 mg/kg and about 2 mg/kg. In yet another embodiment, the dose is between about 0.1 mg/kg and about 5 mg/kg. In still another embodiment, the dose is between about 0.3 mg/kg and about 5 mg/kg. In yet another embodiment, the dose is between about 1 mg/kg and about 5 mg/kg. In still another embodiment, the dose is between about 3 mg/kg and about 5 mg/kg. In particular embodiments, the dose is 0.3 mg/kg or 1.0 mg/kg or 3.0 mg/kg.

Alfimeprase can be co-administered with other drugs, including, but not limited to, anti-coagulants and platelet aggregation inhibitors (anti-platelet drugs). Examples of drugs that can be co-administered with alfimeprase include direct or indirect thrombin inhibitors, Factor Xa inhibitors, P2Y12 antagonists, glycoprotein IIb/IIIa inhibitors, thromboxane inhibitors. In particular embodiments the drug is heparin, a low molecular weight heparin, a heparinoid, fondaparinux, warfarin, dabigatran, bivalirudin, argatroban, rivaroxaban, apixaban, clopidogrel, prasugrel, ticagrelor, tirofiban, eptifibatide, abciximab, or aspirin.

### EXAMPLES

### Example 1

A patient presents with 2 hours of severe chest pain and nausea to his local emergency room. The ECG reveals extensive ST elevation in the precordial leads, and acute anterolateral STEMI is diagnosed. The patient is administered 325 mg of aspirin and taken emergently to cardiac catheterization laboratory. Angiography revealed occlusion of the proximal left anterior descending artery and a large thrombus burden. Alfimeprase is administered directly into the thrombus prior to aspiration thrombectomy. TIMI-3 flow is restored, and patient has relief of pain. Minimal residual thrombus is still present. A 4.0×20 bare metal stent is deployed at 14 atmosphere pressure. There is no residual stenosis and normal flow is observed. The patient is discharged from the hospital after 48 hours.

### Example 2

A patient presents after 6 hours of chest pain and shortness of breath. An ECG reveals extensive ST elevation in the precordial leads and acute anterolateral STEMI is diagnosed. The patient is administered oxygen and aspirin and taken emergently to cardiac catheterization laboratory. Angiography revealed occlusion of the proximal left anterior descending artery and a large thrombus burden. Alfimeprase is administered directly to the thrombus prior to and during angioplasty using a microporous angioplasty catheter. Alfimeprase administration is continued until all the thrombus is cleared. No stenting is performed since there is no residual stenosis. The patient is placed on a maintenance dose of aspirin and clopidogrel and released from the hospital after observation for 48 hours.

What is claimed is:

1. A method of treating a patient with at least one of an arterial or venous thrombus, comprising administering alfimeprase locally to the thrombus.

2. The method according to claim 1, wherein the administration of alfimeprase reduces at least one of a risk of no-reflow, a risk of bleeding, or a risk of distal embolization.

3. The method according to claim 1 further comprising administering alfimeprase to patients experiencing at least one of an ST elevation myocardial infarction or a stroke.

4. The method according to claim 3, further comprising administering alfimeprase within 24 hours of an onset of the ST elevation myocardial infarction.

5. The method according to claim 1, further comprising administering alfimeprase at a dose between 0.01 mg/kg and 20 mg/kg.

6. The method according to claim 1, further comprising administering alfimeprase to at least one of directly into the thrombus or adjacent to the thrombus.

7. The method according to claim 1, further comprising administering alfimeprase using at least one of a catheter, an infusion wire, a balloon, a stent, a drug coated stent, or a drug coated balloon.

8. The method according to claim 1 further comprising administering alfimeprase in conjunction with at least one of removing the thrombus or partially removing the thrombus.

9. The method according to claim 1, further comprising administering alfimeprase with at least one of an anticoagulant or an antiplatelet agent

10. The method according to claim 9, wherein administering the anticoagulant further comprises administering at least one of a direct thrombin inhibitor, an indirect thrombin inhibitor, or a Factor Xa inhibitor.

11. The method according to claim 10, wherein administering the direct thrombin inhibitor further comprises administering at least one of bivalirudin, argatroban, or dabigatran.

12. The method according to claim 10, wherein administering the indirect thrombin inhibitor further comprises administering at least one of heparin, heparinoid, or low molecular weight heparin.

13. The method according to claim 10, wherein administering the Factor Xa inhibitor further comprises administering at least one of apixaban, fondaparinux, or rivaroxaban.

14. The method according to claim 9, wherein administering the antiplatelet agent further comprises administering at least one of a glycoprotein IIb/IIIa inhibitor, a P2Y12 antagonist, or a thromboxane inhibitor.

15. The method according to claim 14, wherein administering the glycoprotein IIb/IIIa inhibitor further comprises administering at least one of abciximab, eptifibatide, or tirofiban.

16. The method according to claim 14, wherein administering the P2Y12 antagonist further comprises administering at least one of clopidogrel, prasugrel, or ticagrelor.

17. The method according to claim 1, further comprising administering alfimeprase in conjunction with at least one of performing an endovascular intervention or performing a mechanical intervention.

18. The method according to claim 17, wherein performing the endovascular intervention further comprises performing at least one of a percutaneous coronary intervention, a thrombectomy, crossing the thrombus with a wire, crossing the thrombus with an infusion wire, a balloon angioplasty, crossing the thrombus with a deployment of a stent, or crossing the thrombus with a catheter.

19. The method according to claim 17, wherein performing the endovascular intervention further comprises deploying a device to at least one of prevent or capture debris.

20. The method according to claim 17, wherein performing the mechanical intervention further comprises delivering a force by at least one of laser or ultrasound.
